(19) United States
(12) Patent Application Publication
(10) Pub. No.: US 2026/0248366 A1
(43) Pub. Date: Aug. 27, 2026

(54) SYSTEMS, DEVICES, AND ASSEMBLIES FOR COLLECTING FLUIDS IN MEDICAL DEVICE HANDLES

(71) Applicant: Boston Scientific Scimed, Inc., Maple Grove, MN (US)

(72) Inventors: Kate Beatrice CONCANNON, Brighton, MA (US); Lisa Sangree McCARTHY, Boston, MA (US); Thomas Joseph BIANCHI, Palmer, MA (US); Gabrielle Nicole GRAMZA, Mishawaka, IN (US); Dennis LYNCH, Glanworth (IE); Allyn Narcisse JENSRUD, Milford, MA (US)

(73) Assignee: Boston Scientific Scimed, Inc., Maple Grove, MN (US)

(21) Appl. No.: 19/545,310

(22) Filed: Feb. 20, 2026

Related U.S. Application Data
(60) Provisional application No. 63/761,468, filed on Feb. 21, 2025.

(51) Int. Cl. A61B 1/00; A61B 1/015

(52) U.S. Cl. CPC A61B 1/00066; A61B 1/015

(57) ABSTRACT

A medical device handle may include a handle housing and a hub disposed within the handle housing. The hub may include a first hub portion, a second hub portion, and a fluid receptacle. The fluid receptacle may be formed between the first hub portion and the second hub portion. The fluid receptacle may be configured to collect fluid that leaks from one or more fluid lumens extending at least partially through the handle housing.

### CROSS-REFERENCE TO RELATED APPLICATIONS

This application claims priority to U.S. Provisional Application No. 63/761,468, filed on Feb, 21, 2025, which is incorporated by reference herein in its entirety.

### TECHNICAL FIELD

The present disclosure relates generally to medical systems, devices, and assemblies. More particularly, in some embodiments, the disclosure relates to systems, devices, and assemblies for collecting fluids in medical device handles.

### BACKGROUND

Endoscopes typically include a flexible shaft that extends from a handle. The flexible shaft contains one or more tubes or lumens that extend through at least a portion of the length of the shaft and into the handle, allowing for the passage of various fluids, tools, and electrical components. During endoscopic procedures, fluids (e.g., irrigation solutions, contrast agents, and/or bodily fluids) may be introduced or removed from a subject. The flow of these fluids is typically conveyed through the one or more tubes or lumens within the endoscope's shaft and handle. However, as fluids travel proximally or distally through the tubes or lumens, there is a risk that they may escape their intended conduits. This can result in fluids leaking from various parts of the endoscope, including the handle. Such leakage can create several issues, including compromised sterility, reduced visibility during procedures, equipment damage, and overall decreased efficiency of the endoscopic system. The disclosure may solve one or more of these problems or other problems in the art. The scope of the disclosure, however, is defined by the attached claims and not the ability to solve a specific problem.

### SUMMARY

According to an example, a medical device handle may include a handle housing and a hub disposed within the handle housing. The hub may include a first hub portion, a second hub portion, and a fluid receptacle. The fluid receptacle may be formed between the first hub portion and the second hub portion. The fluid receptacle is configured to collect fluid that leaks from one or more fluid lumens extending at least partially through the handle housing.

Any of the devices disclosed herein may include any of the following features, alone or in any combination. The hub may further include a lumen through the second hub portion. The lumen may be configured to receive the one or more fluid lumens. A proximal portion of the lumen may taper from a proximal end to a distal end of the proximal portion. An opening may extend from an outer surface of the first hub portion to an inner surface of the lumen. The opening may extend through a bridge disposed between the first hub portion and the second hub portion.

The first hub portion may include at least one cutout. The at least one cutout may be configured to receive a complementary feature of the handle housing. The second hub portion may further comprise a cylindrical portion. A first wing and a second wing may extend proximally from the cylindrical portion. The first hub portion may include a first groove and a second groove each extending along an inner surface of the first hub portion. The first groove may be configured to receive the first wing. The second groove may be configured to receive the second wing. A width of the first wing is smaller than a width of the second wing. A rib may be disposed on a proximal-most end of each of the first wing and the second wing.

The second hub portion may include a proximal cylindrical portion and a distal cylindrical portion. An inner diameter of the proximal cylindrical portion may be greater than an inner diameter of the distal cylindrical portion. A proximal-most end of the first hub portion may be distal of or adjacent to a proximal-most end of the second hub portion.

An internal surface of the handle housing may include an internal stop. A distal end of the internal stop may be configured to abut a proximal-most end of the first hub portion. A proximal-most end of the first hub portion may be distal of or adjacent to a proximal-most end of the second hub portion. An internal surface of the handle housing may include an internal stop. A distal end of the internal stop may be configured to abut a proximal-most end of the first hub portion. The first hub portion may include a frustoconically-shaped outer surface.

According to another example, a hub disposed within a handle of a medical device may include a first hub portion, a second hub portion, and a fluid receptacle. The second hub portion may be disposed within the first hub portion. The second hub portion may include a lumen extending through the second hub portion. The lumen may be configured to receive a tubular member comprising at least one lumen. The fluid receptacle may be formed between the first hub portion and the second hub portion. The fluid receptacle may be configured to collect fluid that leaks from the tubular member.

Any of the devices disclosed herein may include any of the following features, alone or in any combination. The hub may further comprise at least one opening extending from an outer surface of the first hub portion to an inner surface of the lumen of the second hub portion. The second hub portion may further comprise a cylindrical portion. A first wing and a second wing may extend proximally from the cylindrical portion. The first hub portion may include a first groove and a second groove each extending along an inner surface of the first hub portion. The first groove may be configured to receive the first wing. The second groove may be configured to receive the second wing.

According to another example, a medical device handle may include a handle housing and a hub disposed within the handle housing. The hub may include a first hub portion having a first wall and a second hub portion disposed within the first hub portion. The second hub portion may have a second wall. The second wall may define a lumen extending through the second hub portion. The lumen may be configured to receive a tubular member comprising at least one lumen. A gap may be defined between the first wall and the second wall. An adhesive may be disposed between the tubular member and the second wall.

Any of the devices disclosed herein may include any of the features previously described, alone or in any combination.

### BRIEF DESCRIPTION OF FIGURES

The accompanying drawings, which are incorporated in and constitute a part of this specification, illustrate various exemplary embodiments and, together with the description, serve to explain the principles of the disclosed embodiments.

FIGS. 1A and 1B illustrate proximal and distal portions, respectively, of an exemplary medical device, according to aspects of this disclosure.

FIG. 2 illustrates a medical system, including the medical device of FIGS. 1A and 1B, according to aspects of this disclosure.

FIG. 3 illustrates an exploded view of aspects of the proximal portion of the medical device of FIGS. 1A and 1B, according to aspects of this disclosure.

FIG. 4 illustrates an internal view of the proximal portion of the medical device of FIG. 1A and 1B, according to aspects of this disclosure.

FIGS. 5A-5E illustrate various views of an exemplary adapter, according to aspects of this disclosure.

FIGS. 6A and 6B illustrate an alternative adapter, according to aspects of this disclosure.

FIG. 7 illustrates a perspective view of an alternative adapter, according to aspects of this disclosure.

FIGS. 8A-8C illustrate an alternative adapter, according to aspects of this disclosure.

FIGS. 9A-9C illustrate an alternative adapter, according to aspects of this disclosure.

FIGS. 10A-10C illustrate an alternative adapter, according to aspects of this disclosure.

FIGS. 11A-11C illustrate an alternative adapter, according to aspects of this disclosure.

FIGS. 12-15 each illustrate further alternative adapters disposed within the distal portion of the medical device of FIGS. 1A and 1B, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In some drawings, arrows labeled “P” and “D” indicate proximal and distal directions, respectively. The term “exemplary” is used in the sense of “example,” rather than “ideal.” The term “approximately,” or like terms (e.g., “substantially”), includes values +/- 10% of a stated value. The term “integrally formed” and like terms refer to when items are formed of a same piece of material (e.g., are jointly molded) as opposed to being formed as separate elements that may be later affixed to one another. Wherever possible, the same or similar reference numbers will be used through the drawings to refer to the same or like parts. Accordingly, between different embodiments, like numbers will be used to refer to like features, with “100” added to each numeral in successive figures.

The following description sets forth exemplary aspects of the present disclosure. It should be recognized, however, that such description is not intended as a limitation on the scope of the present disclosure. Rather, the description also encompasses combinations and modifications to those exemplary aspects described herein. For example, any of the devices disclosed herein may include any of the following features, additionally or alternatively, in any combination.

Leakage of fluid in endoscopic devices can create several issues, including compromised sterility, reduced visibility during procedures, equipment damage, and overall decreased efficiency of the endoscopic system. For example, the occurrence of fluid leaks from endoscope handles can interfere with the operator's grip and control of the device, as well as lead to contamination of the surrounding environment. Additionally, repeated exposure to leaked fluids may cause degradation of the handle materials over time and may negatively impact the use of the endoscope over the course of a procedure.

The disclosed devices and assemblies may assist in inhibiting fluid leaks at the handle. For example, the disclosed devices and assemblies may include an adapter, or a hub, that defines a space to catch, or retain, fluids that leak from the one or more lumens or tubular members that extend through the handle and flexible shaft. The disclosed devices and assemblies may also include materials to absorb the leaked fluids and/or one or more seals to inhibit fluids from leaking from the handle. The disclosed aspects may improve reliability, safety, and effectiveness of endoscopic procedures.

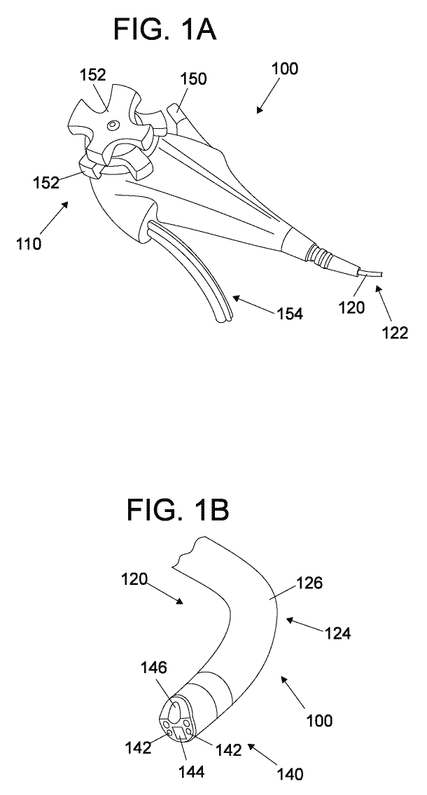

FIGS. 1A and 1B depict proximal and distal portions, respectively, of a first medical device 100. In some examples, first medical device 100 may be a cholangioscope. However, although cholangioscopes are referenced herein, it will be appreciated that first medical device 100 may be another type of daughter scope, an endoscope, a ureteroscope, a duodenoscope, a gastroscope, an endoscopic ultrasonography (“EUS”) scope, a colonoscope, a bronchoscope, a laparoscope, an arthroscope, a cystoscope, an aspiration scope, a sheath, a catheter, a tome, or any type of accessory instrument having a shaft and a handle.

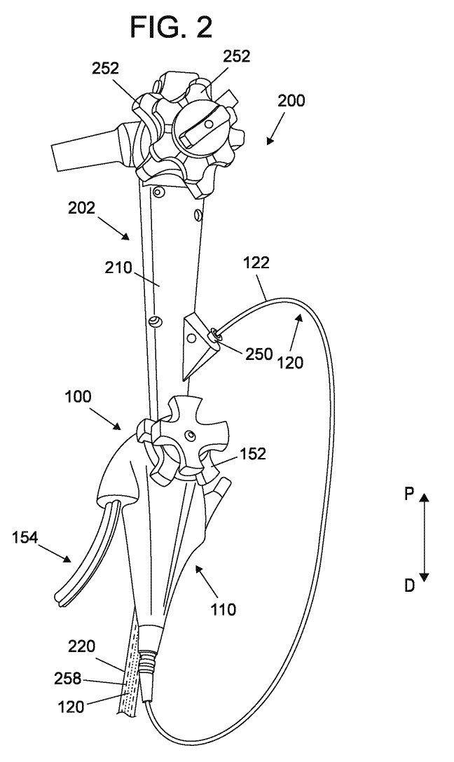

Referring to FIG. 1A, first medical device 100 may include a handle 110 and a shaft 120. Referring to FIG. 1B, a distal tip 140 may be coupled to a distal end of shaft 120. Shaft 120 may be a tubular member having one or more lumens. Shaft 120 may include a proximal portion 122 (FIG. 1A) and a distal portion 124 (FIG. 1B). Discussed in greater detail below, a proximal-most portion 122P of proximal portion 122 of shaft 120 may extend proximally into a portion of handle 110. In some aspects, proximal-most portion 122P may be only a subset of proximal portion 122 of shaft 120 (e.g., a tube inside of proximal portion 122). Distal portion 124 of shaft 120 and distal tip 140 may be configured to be inserted into a body lumen of a subject. For example, distal portion 124 of shaft 120 and distal tip 140 may be inserted into a working channel of a second medical device (e.g., second medical device 202, shown in FIG. 2 and described below). Fluids may be delivered and/or removed from the subject via the working channel or any other lumen of shaft 120. As described in further detail below, proximal portion 122 may remain outside of/external to the body lumen of the subject and the working channel of the second medical device. Distal portion 124 may include a steerable section 126, which may include an articulation joint or other structure (e.g., a multi-lumen extruded shaft or other bendable shaft) that is configured to deflect or otherwise articulate in order to steer steerable section 126 and distal tip 140.

Distal tip 140 may include one or more lighting elements 142, one or more imaging devices 144, and a distal opening 146 of a working channel of first medical device 100. One or more lighting elements 142, one or more imaging devices 144, and distal opening 146 may have any suitable arrangement. Alternatively or additionally, distal tip 140 may include one or more end effectors (e.g., graspers, snares, staplers, clips, stents, forceps, suturing devices, baskets, etc.) for performing a medical procedure at a treatment site.

Handle 110 may include a port 150, which may provide access to the working channel of first medical device 100. An accessory instrument may be inserted into port 150, advanced through the working channel, and out of distal opening 146 of distal tip 140. Handle 110 may also include one or more actuators 152 for controlling aspects of shaft 120 and/or distal tip 140. For example, one or more actuators 152 may include one or more steering knobs (or levers, sliders, etc.) for controlling articulation of steerable section 126. For example, one or more cables and/or wires may extend from one or more actuators 152 to steerable section 126. Tensioning or de-tensioning of the cables and/or wires may cause steerable section 126 to deflect in one or more of an up/down or left/right direction. In alternatives, actuator(s) 152 may be used to control an end effector of distal tip 140 using one or more control wires or cables. Actuator(s) 152 may additionally or alternatively control electronic elements of distal tip 140 and/or deliver air, suction, and/or water.

An umbilicus 154 may extend from handle 110. Umbilicus 154 may include one or more cables, tubes, or other conduits for conveying electrical signals, air, water, and/or suction to or from handle 110, shaft 120, or distal tip 140. Umbilicus 154 may include one or more connectors (not shown) for connecting to capital equipment, which may supply electrical controls, air, water, and/or suction.

FIG. 2 depicts a medical system 200, including first medical device 100 and a second medical device 202. Second medical device 202 may be, for example, a duodenoscope or any other type of medical scope. For example, although a duodenoscope is referenced herein, it will be appreciated that second medical device 202 may be any medical device having a working channel extending from a proximal end to a distal end, such as a ureteroscope, an endoscope, a gastroscope, an endoscopic ultrasonography (“EUS”) scope, a colonoscope, a bronchoscope, a laparoscope, an arthroscope, a cystoscope, an aspiration scope, a sheath, or a catheter.

Second medical device 202 may include a handle 210 and a shaft 220. Although not shown in FIG. 2, a distal tip having any of the features of distal tip 140 may be at a distal end of shaft 220. Shaft 220 may be insertable into a body lumen of a subject in order to perform a procedure at a target site in the body lumen or other portion of the body.

Handle 210 may include a port 250. Port 250 may provide access to a working channel 258 of second medical device 202, (e.g., of shaft 220). A medical device (e.g., first medical device 100) may be configured to be inserted into port 250, advanced through working channel 258 of shaft 220, and extended distally from the distal tip of second medical device 202. Working channel 258 is shown in dotted-lines within shaft 220 to illustrate working channel 258 extending through an interior portion of shaft 220. Handle 210 may also include one or more actuators 252 for controlling aspects of shaft 220 and/or a distal tip of second medical device 202. For example, actuators 252 may include one or more knobs, levers, sliders, joysticks, buttons, or the like for articulating a distal portion of shaft 220. One or more actuators 252 further include levers, knobs, buttons, sliders, buttons, or the like for locking the distal portion of shaft 220 from articulating. One or more actuators 252 may also include one or more actuators for capturing images from an imaging device of a distal tip of second medical device 202, or for delivering air, water, and/or suction to a distal tip of second medical device 202.

As shown in FIG. 2, handle 110 of first medical device 100 may be coupled to handle 210 of second medical device 202. For example, a strap, clamp, or the like may couple handle 110 of first medical device 100 to handle 210 of second medical device 202. Distal tip 140 may be inserted into port 250, and distal portion 124 of shaft 120 may be advanced through shaft 220 until distal tip 140 extends from a distal tip of second medical device 202. Because shaft 220 may be inserted into a body lumen of a subject, distal portion 124 of shaft 120 may also be inserted into the body lumen of the subject.

During use, first medical device 100 may be oriented such that a longitudinal axis of the handle is approximately perpendicular to the ground, for example, as shown in FIG. 2. For example, first medical device 100 may be held by a user or first medical device 100 may be coupled to second medical device 202 such that first medical device 100 is approximately perpendicular to the ground. Accordingly, during use, fluids may leak from any portion of handle 110 of first medical device 100, and particularly from or within a distal end of handle 110 of first medical device.

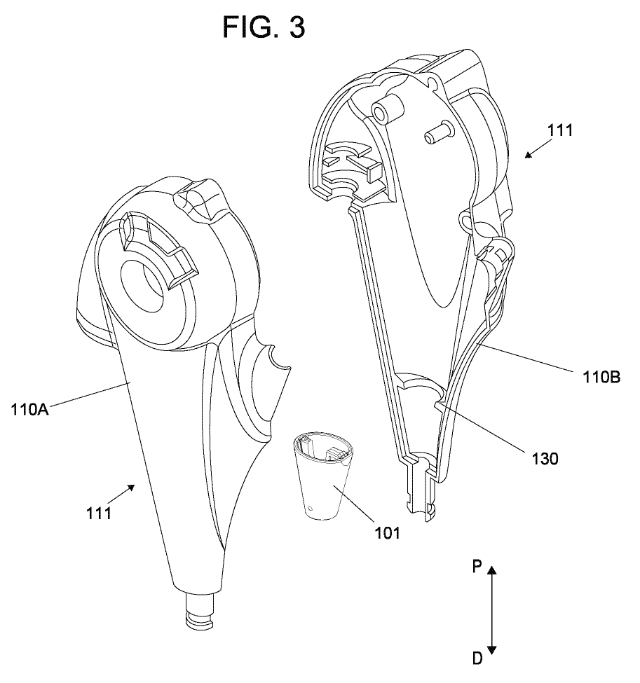

FIG. 3 illustrates an exploded view of aspects of handle 110 of first medical device 100. Handle 110 may include a housing 111 having a first handle housing portion 110A and a second handle housing portion 110B that are configured to mate together to form a handle assembly. A hub 101 may be positioned between first handle housing portion 110A and second handle housing portion 110B, for example, within a distal end portion of handle 110. Hub 101 may be designed to fit between first handle housing portion 110A and second handle housing portion 110B, allowing for proper positioning and retention within the assembled handle housing 111.

In some cases, second handle housing portion 110B may include at least one internal stop 130. The configuration of internal stop 130 within second handle housing portion 110B may provide a mechanism for controlling the position of hub 101 within handle 110 and inhibiting undesired movement during use of first medical device 100. For example, internal stop 130 may be a protrusion or ledge that extends radially inward from an inner surface of second handle housing portion 110B. In some aspects, although not shown due to the orientation of first handle housing portion 110A on the page, first handle housing portion 110A may also include an internal stop 130 extending radially inward from an inner surface of first handle housing portion 110A. For example, internal stop 130 may be proximal of a proximal-most end of hub 101. Internal stop 130 may interact with, or abut, the proximal-most end of hub 101. In some aspects, internal stop 130 may be a chamfer, for example, configured to direct fluid towards hub 101. For example, internal stop 130 may include surface(s) angled towards hub 101. Accordingly, fluid may be directed towards hub 101. Such a configuration of internal stop 130 may be beneficial, for example, when handle 110 is held at an angle less than 90 degrees relative to the ground (e.g., such that a longitudinal axis of handle 110 is approximately parallel to the ground).

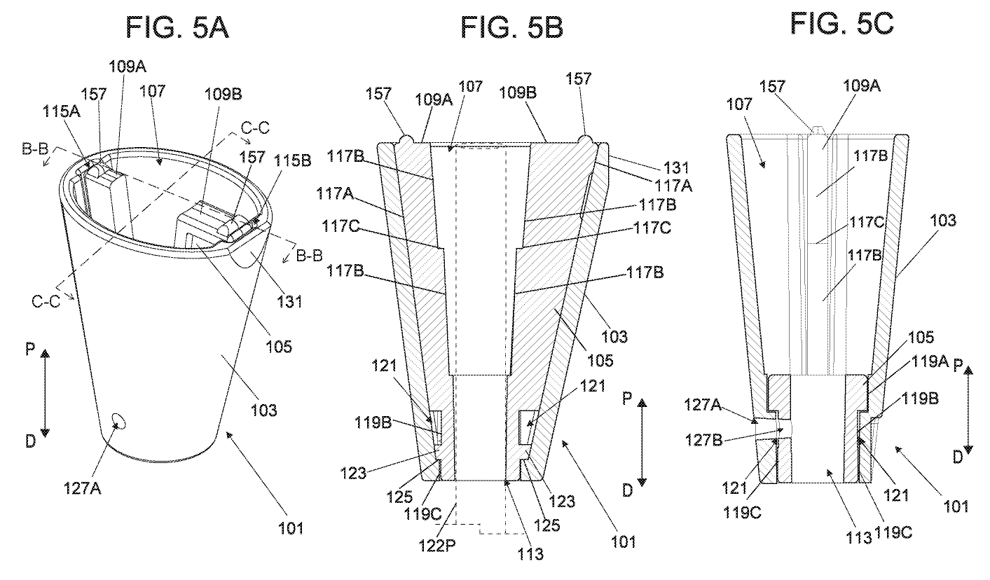

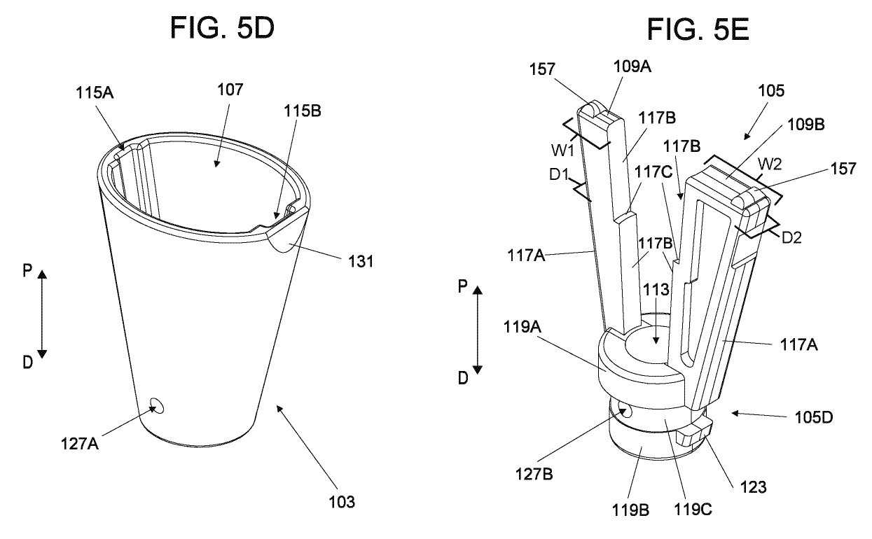

Although not shown, each of first handle housing portion 110A and second handle housing portion 110B may include additional protrusions and/or indentations disposed on the internal surface of each respective handle housing portion 110A, 110B. Such protrusions and/or indentations may be configured to interact with hub 101, for example, to assist in maintaining a position of hub 101 within handle 110 (e.g., to inhibit proximal or rotational movement of hub 101 within handle 110). Hub 101 may include complementary features (e.g., indentations and/or protrusions) to receive the features of a respective handle housing portion 110A, 110B. Additional aspects of hub 101 are shown and described below with respect to FIGS. 5A-5E.

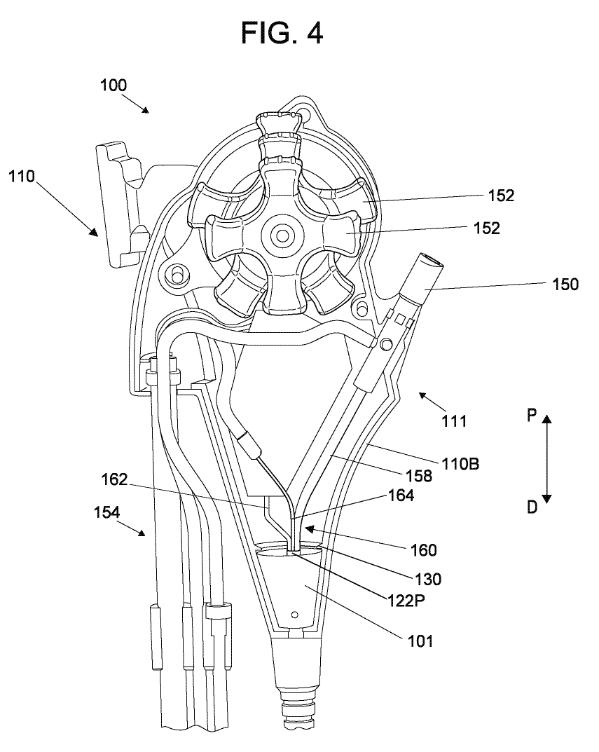

FIG. 4 illustrates a portion of handle 110 of first medical device 100, with portions of first handle housing portion 110A transparent. As described with respect to FIG. 3, hub 101 may be positioned within second handle housing portion 110B and may interface with internal stop 130. One or more actuators 152 may be positioned on the exterior of first handle housing portion 110A, allowing for control of various functions of first medical device 100, including, for example, articulation of steerable section 126 (FIG. 1B) of first medical device 100.

Umbilicus 154 may extend from a proximal portion of handle 110. A plurality of members 160 may extend from handle 110, port 150, and/or umbilicus 154, and into, or through, proximal-most portion 122P of shaft 120. Members 160 may include working channel 158, camera or electrical cables/wire(s) 162, and/or other fluidics tube(s) 164. Members 160, including, for example working channel 158 and fluidics tubes 164 may be coupled to or extend at least partially into proximal-most portion 122P of shaft 120 (e.g., into respective lumens of proximal-most portion 122P). For example, working channel 158 may extend from port 150 to/into proximal-most portion 122P shaft 120. Proximal-most portion 122P of shaft 120 may extend through hub 101.

The internal configuration shown in FIG. 4 may allow for the integration of fluid management features, such as hub 101, within the compact space of housing 111 of handle 110. The arrangement of hub 101 within housing 111 of handle 110 may help inhibit fluid leakage from handle 110 while maintaining the functionality of first medical device 100.

FIGS. 5A-5E each illustrate various views of hub 101. For example, FIG. 5A illustrates a perspective view of hub 101; FIG. 5B illustrates a cross-sectional view of hub 101 taken at the plane labeled B-B line in FIG. 5A; FIG. 5C illustrates an alternative cross-sectional view of hub taken at the plane labeled C-C in FIG. 5A; FIG. 5D illustrates a perspective view of a first hub portion 103 of hub 101; and FIG. 5E illustrates a perspective view of a second hub portion 105 of hub 101. In particular, the cross-sectional view of hub 101 shown in FIG. 5B is through a widest diameter of hub 101 shown in FIG. 5A. The cross-sectional view of hub 101 shown in FIG. 5C is through a narrowest diameter of hub 101 shown in FIG. 5A. All of FIGS. 5A-5E may be referred to in the following description.

Hub 101 may include a first, outer hub portion 103 and a second, inner hub portion 105. First hub portion 103 of hub 101 may be configured to receive second hub portion 105. Second hub portion 105 may be fixed to proximal portion 120P of first medical device 100. For ease of illustration, proximal portion 120P is shown in broken lines only in FIG. 5B. First hub portion 103 may be frustoconically-shaped. For example, a proximal end of first hub portion 103 of hub 101 may be wider (e.g., have a greater diameter) as compared to a distal end of first hub portion 103 of hub 101. First hub portion 103 may be complementarily shaped so as to fit within a distal portion of handle 110 (e.g., between first handle housing portion 110A and second handle housing portion 110B). In aspects, a proximal end of first hub portion 103 may include one or more features 131. Such features 131 may be configured to mate with complementary features of handle 110. For example, features 131 may include cutouts, protrusions, and/or indentations configured to mate with complementary features of handle 110. Such features 131 may be configured to assist with the alignment and assembly of hub 101 within handle 110. In other aspects, features 131 may be molding features (e.g., features resulting from the molding process of hub 101).

Hub 101 may define a fluid receptacle 107 formed by first hub portion 103 and second hub portion 105. Fluid receptacle 107 may be configured to hold, or contain, fluids that leak from proximal-most portion 122P of shaft 120, working channel 158, and/or fluidics tubes 164. For example, during use, fluids may leak from a proximal-most end of proximal portion 120P, working channel 158, fluidics tubes 164, a junction formed between various members (e.g., junctions at, near, or within proximal-most portion 122P, with port 150, among working channel 158 and fluidics tubes 164, or between valves and fluidics tubes 164), The fluids may fall into or be contained by fluid receptacle 107 of hub 101. In these aspects, fluid leakage from handle 110 may be inhibited.

Second hub portion 105 of hub 101 may include a first wing 109A and a second wing 109B. Each wing 109A, 109B may taper such that a width of each wing 109A, 109B is greater at a proximal-most end as compared to a distal-most end of each wing 109A, 109B. Outward-facing surfaces 117A of each wing may taper inward (e.g., towards the longitudinal axis of hub 101) from the proximal-most end to the distal-most end. Inward-facing surfaces 117B of each wing 109A, 109B may taper smoothly inward (e.g., towards the longitudinal axis of hub 101) from the proximal-most end to the distal-most end. Additionally or alternatively, inward-facing surfaces 117B of each wing 109A, 109B may be tiered such that a ledge 117C is defined between at least two tiered portions of inward-facing surfaces 117B. The tiered inward-facing surfaces 117B of each wing 109A, 109B may assist in creating a greater distance between each wing 109A, 109B at or near the proximal end of each wing 109A, 109B and a smaller distance between each wing 109A, 109B at or near the distal end of each wing 109A, 109B. A greater distance between the proximal-most ends of each wing 109A, 109B may be advantageous, for example, proximal-most portion 122P of shaft 120 to second hub portion 105. For example, the greater distance between each wing 109A, 109B may permit a user to more easily visualize and grasp or adjust proximal-most portion 122P of shaft 120 through second hub portion 105. As discussed above, however, ledge 117C may be omitted.

Shown more clearly in FIG. 5E, a largest width, W1, of first wing 109A may be smaller than a largest width, W2, of second wing 109B. A largest width of each of first wing 109A and second wing 109B may be measured from inward-facing surfaces 117B of each wing 109A, 109B to the outward-facing surface 117A of each wing 109A, 109B. Similarly, first wing 109A may have a smaller depth, D1, as compared to a depth, D2, of second wing 109B. The depths of each wing 109A, 109B may be measured from a front wall of each wing 109A, 109B to a back wall of each wing 109A, 109B. In other words, second wing 109B may be thicker as compared to first wing 109A.

The different dimensions of first wing 109A as compared to second wing 109B may assist with assembly of hub 101. For example, first hub portion 103 may include a first groove 115A and a second groove 115B, each defined on an internal surface of first hub portion 103. Grooves 115A, 115B may be referred to as rails or slots and may each extend from a proximal end of first hub portion 103 to a distal portion of first hub portion 103. First groove 115A may be sized and shaped to only receive first wing 109A, and second groove 115B may be sized and shaped to receive second wing 109B. For example, first groove 115A may be narrower than second groove 115B. In these aspects, second hub portion 105 may only be inserted into first hub portion 103 a single way. Thus, grooves 115A, 115B may assist in inhibiting second hub portion 105 from being inserted into first hub portion 103 incorrectly.

A proximal-most end of one or both wings 109A, 109B may include a protrusion 157 extending proximally. Each protrusion 157 may be a crush rib, configured to interact with internal stop 130 of first handle housing portion 110A and/or second handle housing portion 110B (shown in FIGS. 3 and 4). Protrusion 157 may be configured to deform or bend during the assembly of hub 101 within handle 110. For example, during assembly of hub 101 within handle 110 (e.g., within first handle housing portion 110A or second handle housing portion 110B), protrusion 157 may interact with internal stop 130 and deform. The deformation of protrusion 157 may assist in creating an interference fit against internal stop 130 such that hub 101 is snuggly disposed within handle 110 (e.g., within first handle housing portion 110A and/or second handle housing portion 110B). In other aspects, internal stop 130 may include complementary grooves or indentations to receive protrusion 157 on one or both wings 109A, 109B.

Each wing 109A, 109B may extend proximally from a distal portion 105D of second hub portion 105. For example, distal portion 105D of second hub portion 105 may include a first, proximal cylindrical portion 119A, a second, distal cylindrical portion 119B, and a central cylindrical portion 119C disposed between proximal cylindrical portion 119A and distal cylindrical portion 119B. Each of proximal cylindrical portion 119A, distal cylindrical portion 119B, and central cylindrical portion 119C may have a different diameter. A diameter of proximal cylindrical portion 119A may be greater than diameters of each of distal cylindrical portion 119B and central cylindrical portion 119C, and a diameter of central cylindrical portion 119C may be smaller than a diameter of each of proximal cylindrical portion 119A and distal cylindrical portion 119B. In other words, central cylindrical portion 119C may be recessed relative to proximal cylindrical portion 119A and distal cylindrical portion 119B. When second hub portion 105 is assembled with first hub portion 103, a gap 121 may be defined between first hub portion 103 and second hub portion 105.

In some aspects, distal cylindrical portion 119B may include one or more protrusions 123 extending radially outward. Protrusion(s) 123 may be configured to interact with a ledge 125 of first hub portion 103, e.g., so as to inhibit second hub portion 105 from traveling distally relative to first hub portion 103. Protrusion(s) 123 may be configured to ensure proper longitudinal alignment of second hub portion 105 within first hub portion 103. In aspects, protrusions 123 may be omitted.

During assembly, second hub portion 105 may be fixed to proximal-most portion 122P of shaft 120 using, for example, an adhesive and/or an interference fit. For example, proximal-most portion 122P of shaft 120 may be inserted through central lumen 113. A proximal-most end of proximal-most portion 122P of shaft 120 may terminate distally of a proximal-most end of hub 101. For example, the proximal-most end of working channel 158 may extend between first wing 109A and second wing 109B. In aspects, the proximal-most end of working channel 158 may terminate between first wing 109A and second wing 109B. For ease of illustration, working channel 158 is shown in broken lines in FIG. 5B. Proximal-most portion 122P of shaft 120 may not contact first wing 109A and second wing 109B, such that a gap is formed between proximal-most portion 122P of shaft 120 and first wing 109A and second wing 109B.

In some aspects, an adhesive may be injected through an opening 127B defined within second hub portion 105 to secure proximal-most portion 122P of shaft 120 within central lumen 113. Opening 127B may be defined through an outer wall of distal portion 105D and opening 127B may be in fluid communication with central lumen 113. The adhesive may flow into central lumen 113 defined within distal portion 105D of second hub portion 105. The adhesive flowing into central lumen 113 may assist in fixing a portion of proximal-most portion 122P of shaft 120 within central lumen 113 and, thus, to second hub portion 105. The second hub portion 105 may then be assembled with first hub portion 103. For example, an adhesive may be injected through an opening 127A defined within first hub portion 103. The adhesive may flow into gap 121 to assist with fixing second hub portion 105 within first hub portion 103. Hub 101 may then be loaded into handle 110.

In other aspects, first hub portion 103 and second hub portion 105 may be first assembled such that openings 127A, 127B of each hub portion 103, 105, respectively are aligned with one another. Proximal-most portion 122P of shaft 120 may then be loaded into second hub portion 105. An adhesive may then be injected through openings 127A, 127B. The adhesive may flow into gap 121 to fix first hub portion 103 to second hub portion 105. The adhesive may also flow into central lumen 113 to fix working channel 158 to second hub portion 105. Hub 101 may then be loaded into handle 110.

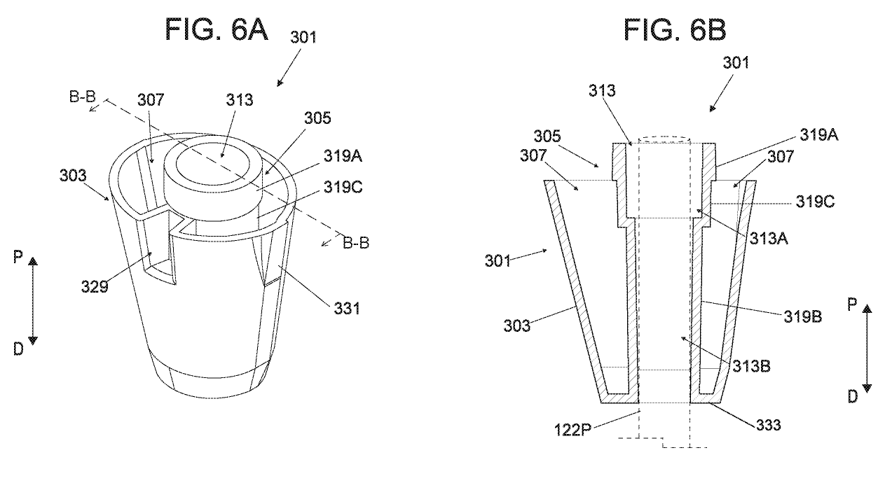

FIGS. 6A and 6B illustrate an alternative exemplary hub 301. For example, FIG. 6A illustrates a perspective view of hub 301, and FIG. 6B illustrates a cross-sectional view of hub 301 taken at the plane labeled B-B in FIG. 6A. Hub 301 may have any or all of the characteristics of hub 101, described above, except as described below. For example, hub 301 may be used with handle 110 of first medical device 100.

Hub 301 may include a first hub portion 303 and a second hub portion 305. In aspects, first hub portion 303 may be integrally formed with second hub portion 305 from a same piece of material (e.g., may be molded together). For example, a distal wall 333 may define a distal-most end of hub 301 and serve to connect first hub portion 303 with second hub portion 305. Second hub portion 305 may be fixed to proximal-most portion 122P of shaft 120 of first medical device 100. For ease of illustration, proximal-most portion 122P of shaft 120 is shown in broken lines only in FIG. 6B.

Hub 301 may define a fluid receptacle 307. Fluid receptacle 307 may at least partially circumferentially surround second hub portion 305. In other words, second hub portion 305 may be disposed within first hub portion 303, and fluid receptacle 307 may be defined between first hub portion 303 and second hub portion 305. Fluid receptacle 307 may be configured to hold, or contain, fluids that leak from proximal-most portion 122P of shaft 120, working channel 158, and/or fluidics tubes 164. In these aspects, hub 301 may be configured to inhibit leaks from handle 110.

Second hub portion 305 may include a first, proximal cylindrical portion 319A, a second, distal cylindrical portion 319B, and a central cylindrical portion 319C disposed therebetween. Each of proximal cylindrical portion 319A, distal cylindrical portion 319B, and central cylindrical portion 319C may have a different diameter. A diameter of proximal cylindrical portion 319A may be greater than diameters of each of distal cylindrical portion 319B and central cylindrical portion 319C, and a diameter of central cylindrical portion 319C may be smaller than a diameter of each of proximal cylindrical portion 319A and distal cylindrical portion 319B.

An inner surface of each first cylindrical portion 319A, second cylindrical portion 319B, and central cylindrical portion 319C may define a central lumen 313. Central lumen 313 may be configured to receive proximal-most portion 122P of shaft 120. A diameter of a proximal portion 313A of central lumen 313 may be greater than a diameter of a distal portion 313B of central lumen 313. In other words, an inner diameter of each of first cylindrical portion 319A and central cylindrical portion 319C (which may form proximal portion 313A of central lumen 313) may be approximately equal to one another and greater than an inner diameter of second cylindrical portion 319 (which may form distal portion 313B of central lumen 313). The greater diameter of proximal portion 313A of central lumen 313 may create a well, or a gap, around a proximal end of proximal-most portion 122P of shaft 120. Accordingly, a user may inject or apply adhesive around a portion of proximal-most portion 122P of shaft 120 and within the well created by proximal portion 313A of central lumen 313. The adhesive may assist with fixing proximal-most portion 122P of shaft 120 within second hub portion 305, and thus, to hub 301.

Shown more clearly in FIG. 6A, hub 301 may include a cutout 329, or a notch, at or near a proximal end of first hub portion 103. Cutout 329 may extend radially inward, for example, from an outermost surface of first hub portion 303 to an outward-facing surface of second hub portion 305 (e.g., an outward-facing surface of central cylindrical portion 319C). Cutout 329 may be configured to receive a protrusion extending from an internal surface of first handle housing portion 110A or second handle housing portion 110B (described above with respect to FIG. 3). Upon receipt of the protrusion from the first handle housing portion 110A or second handle housing portion 110B, cutout 329 may be configured to inhibit movement (e.g., rotational movement or proximal movement) of hub 301 within handle 110. In these aspects, cutout 329 may be configured to assist with the alignment and positioning of hub 301 within handle 110.

First hub portion 303 may also include a protrusion 331 at or near a proximal-most end of first hub portion 303 of hub 301. Protrusion 331 may be configured to mate with complementary features (e.g., recesses) of handle 110. In aspects, protrusion 331 may be configured to assist with the alignment and assembly of hub 101 within handle 110. Protrusion 331 may be on a single side of first hub portion 303, or on both sides of first hub portion 303 (e.g., opposite to one another and/or approximately 90 degrees relative to cutout 329).

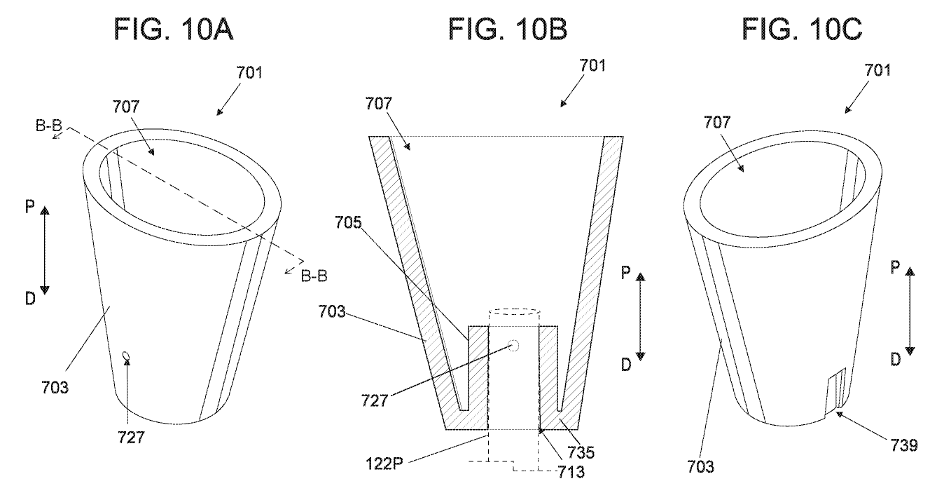

During assembly, proximal-most portion 122P of shaft 120 may be inserted through central lumen 313. A proximal-most end of proximal-most portion 122P of shaft 120 may terminate adjacent to or proximal of a proximal-most end of second hub portion 305 of hub 301. An adhesive may be applied within proximal portion 313A of central lumen 313 to fix proximal-most portion 122P of shaft 120 to hub 301. Hub 301 may then be assembled into handle 110. With second hub portion 305 extending proximally relative to first hub portion 303, proximal-most portion 122P of shaft 120 may be visible from a proximal end of hub 301 (e.g., when inserted through central lumen 413). Visibility of proximal-most portion 122P of shaft 120 from the proximal end of hub 301 may allow for easier assembly. Furthermore, a length of second hub portion 305 may provide for additional surface area within central lumen 313 to which proximal-most portion 122P may be fixed (e.g., as compared to hub 701 of FIGS. 10A-10C, discussed below).

During use, fluids may leak from proximal-most portion 122P of shaft 120 and/or other fluidics tubes 164. The leaked fluid may be collected within fluid receptacle 307 of hub 301. Accordingly, leaks from handle 110 may be inhibited.

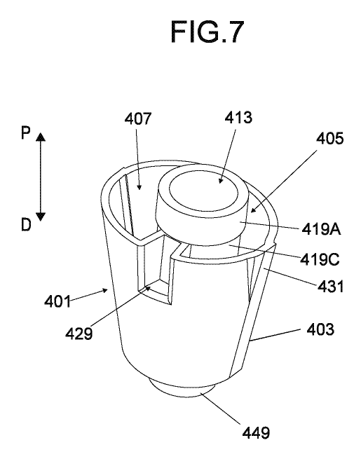

FIG. 7 illustrates a perspective view of an alternative exemplary hub 401. Hub 401 may have any or all of the same characteristics of hub 101 and/or hub 301, except as described below. For example, hub 401 may be used with handle 110 of first medical device 100.

Similar to hub 301, hub 401 may include a first hub portion 403 and a second hub portion 405. In aspects, first hub portion 403 may be integrally formed with second hub portion 405 from a single piece of material (e.g., may be jointly molded). Second hub portion 405 may be fixed to proximal-most portion 122P of shaft 120 of first medical device 100, similar to how hub 301 may be fixed to proximal-most portion 122P of shaft 120 (described above with respect to FIG. 6B). Hub 401 may define a fluid receptacle 407. Fluid receptacle 407 may at least partially circumferentially surround second hub portion 405. Fluid receptacle 407 may be configured to hold, or contain, fluids that leak from proximal-most portion 122P of shaft 120, working channel 158, and/or fluidics tubes 164. In these aspects, hub 401 may be configured to inhibit leaks from handle 110.

Second hub portion 405 may have any or all of the characteristics of second hub portion 305 of hub 301. In particular, second hub portion 405 may include a first, proximal cylindrical portion 419A, a second, distal cylindrical portion (not shown), and a central cylindrical portion 419C disposed therebetween. A central lumen 413 may extend proximally to distally through proximal cylindrical portion 419A, distal cylindrical portion, and central cylindrical portion 419C. The inner and outer diameters of each cylindrical portion may have the same characteristics as the cylindrical portions 319A, 319B, 319C of hub 301, described above with respect to FIGS. 6A and 6B. In these aspects, central lumen 413 may have any or all of the same characteristics of central lumen 313, as described above with respect to hub 301 of FIGS. 6A and 6B.

Hub 401 may include a cutout 429, or a notch. Cutout 429 may have any or all of the characteristics of cutout 329, described above with respect to hub 301 of FIGS. 6A and 6B. Similarly, first hub portion 403 may include a protrusion 431 at or near a proximal-most end of first hub portion 403 of hub 401. Protrusion 431 may have any or all of the same characteristics of protrusion 331, described above with respect to hub 301 of FIG. 6A. Cutout 429 and/or protrusion 431 may be configured to receive or be received by complementary features of handle 110. In these aspects, cutout 429 and/or protrusion 431 may be configured to assist with the placement and/or alignment of hub 401 within handle 110.

Hub 401 may include a distal protrusion 449 extending from a distal-most end of first hub portion 403. Distal protrusion 449 may have a smaller diameter as compared to first hub portion 403. In aspects, distal protrusion 449 may be coaxial with central lumen 413, and central lumen 413 may extend through distal protrusion 449. Distal protrusion 449 may assist with alignment and placement of hub 401 within handle 110. For example, the internal surface of handle 110 may include features to receive distal protrusion 449.

In other aspects, a sealing member (e.g., an O-ring, a seal, a gasket, etc.) may be disposed around distal protrusion 449. With hub 401 disposed within handle 110, sealing member may be configured to press against the internal surfaces of handle housing portions 110A, 110B, thus creating a seal between hub 401 and handle housing portions 110A, 110B. The seal may assist in inhibiting leaks from handle 110.

Proximal-most portion 122P of shaft 120 may be fixed to central lumen 413 similarly to how proximal-most portion 122P of shaft 120 may be fixed to central lumen 313 of hub 301. With second hub portion 405 extending proximally relative to first hub portion 403, proximal-most portion 122P of shaft 120 may be visible from a proximal end of hub 401 (e.g., when inserted through central lumen 413). Visibility of proximal-most portion 122P of shaft 120 from the proximal end of hub 401 may allow for easier assembly. Furthermore, a length of second hub portion 405 may provide for additional surface area within central lumen 413 to which proximal-most portion 122P may be fixed (e.g., as compared to hub 701 of FIGS. 10A-10C, discussed below).

During use, fluids may leak from proximal-most portion 122P of shaft 120 and/or other fluidics tubes 164. The leaked fluid may be collected within fluid receptacle 407 of hub 401. Accordingly, leaks from handle 110 may be inhibited.

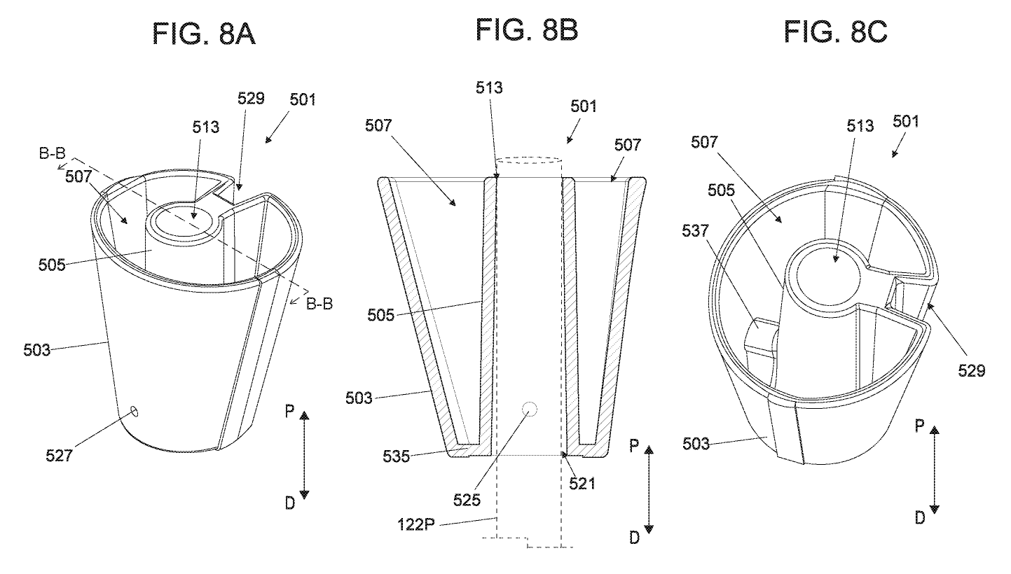

FIGS. 8A-8C illustrate an alternative hub 501. In particular, FIG. 8A illustrates a first perspective view of hub 501, FIG. 8B illustrates a cross-sectional view of hub 501 taken at the plane labeled B-B in FIG. 8A, and FIG. 8C illustrates a top perspective view of hub 501. Hub 501 may be used with handle 110 of first medical device 100. Hub 501 may have any or all of the characteristics of hub 101, hub 301, and/or hub 401, except as described below.

Hub 501 may include a first hub portion 503 and a second hub portion 505. In aspects, first hub portion 503 may be integrally formed with second hub portion 505, for example, via a distal wall 535 (FIG. 8B) extending between first hub portion 503 and second hub portion 505. A proximal-most end of each of first hub portion 503 and second hub portion 505 may be longitudinally aligned.

Hub 501 may define a fluid receptacle 507. Fluid receptacle 507 may at least partially circumferentially surround second hub portion 505. Fluid receptacle 507 may be configured to hold, or contain, fluids that leak from proximal-most portion 122P of shaft 120, working channel 158, and/or fluidics tubes 164. In these aspects, hub 501 may be configured to inhibit leaks from handle 110. As compared with some other embodiments, fluid receptacle 507 may have a larger volume.

Hub 501 may include a central lumen 513 extending proximally to distally through second hub portion 505. Central lumen 513 may be configured to receive proximal-most portion 122P of shaft 120. In these aspects, second hub portion 505 may be fixed to proximal-most portion 122P of shaft 120. For ease of illustration, proximal-most portion 122P of shaft 120 is shown in broken lines only in FIG. 8B. A proximal-most end of proximal-most portion 122P of shaft 120 may terminate adjacent to or proximal of a proximal-most end of second hub portion 505. Central lumen 513 may be tapered such that a diameter of a proximal end of central lumen 513 is smaller than a diameter of a distal end of central lumen 513. A side opening 527 may extend from an outermost surface of first hub portion 503 to an inner surface of second hub portion 505 (e.g., to an inner surface defining central lumen 513). Side opening 527 may be in fluid communication with central lumen 513.

To ensure a continuous opening from first hub portion 503 to second hub portion 505 (e.g., an opening that extends into central lumen 513), side opening 527 may extend through a bridge portion 537. Bridge portion 537 may be defined within a proximal portion of hub 501. Bridge portion 537 may be a wall that extends between first hub portion 503 and second hub portion 505, for example, through fluid receptacle 507. Side opening 527 may extend continuously through bridge portion 537. Side opening 527 may be configured to permit the injection of an adhesive into central lumen 513, for example, to assist with fixing proximal-most portion 122P of shaft 120 to second hub portion 505 of hub 501 (e.g., within central lumen 513). Opening 527 (and central lumen 513) may be isolated from fluid receptacle 507, such that adhesive injected into opening 527 does not flow into fluid receptacle 507.

First hub portion 503 may include a cutout 529, or a notch. Cutout 529 may have any or all of the characteristics of cutout 329 of hub 301 and/or cutout 429 of hub 401, described above. In aspects, cutout 529 may be only at a distal portion of hub 501. In other aspects, cutout 529 may extend from a proximal-most end of hub 501 to a distal-most of hub 501. Cutout 529 may be configured to receive a complementary feature of handle 110, for example, to assist with the placement and/or alignment of hub 501 within handle 110.

To assemble hub 501, proximal-most portion 122P of shaft 120 may be inserted through central lumen 513 and fixed to hub 501 by injecting an adhesive into central lumen 513 via side opening 527. The adhesive may fill a gap 521 defined between proximal-most portion 122P of shaft 120 and a distal portion of central lumen 513, for example, due to the tapered shape of central lumen 513. Filling gap 521 on the distal end of hub 501 (e.g., as opposed to the proximal end of hub) may inhibit adhesive from overflowing into fluid receptacle 507 and/or lumens of proximal-most portion 122P of shaft 120. In aspects, proximal-most portion 122P of shaft 120 may be visible from a proximal end of hub 501 when inserted through central lumen 513. Visibility of proximal-most portion 122P of shaft 120 from the proximal end of hub 501 may allow for easier assembly. Furthermore, a length of second hub portion 505 may provide for additional surface area within central lumen 513 to which proximal-most portion 122P may be fixed (e.g., as compared to hub 701 of FIGS. 10A-10C, discussed below).

During use, fluids may leak from proximal-most portion 122P of shaft 120, working channel 158, and/or other fluidics tubes 164. The leaked fluid may be collected within fluid receptacle 507 of hub 501. Accordingly, leaks from handle 110 may be inhibited.

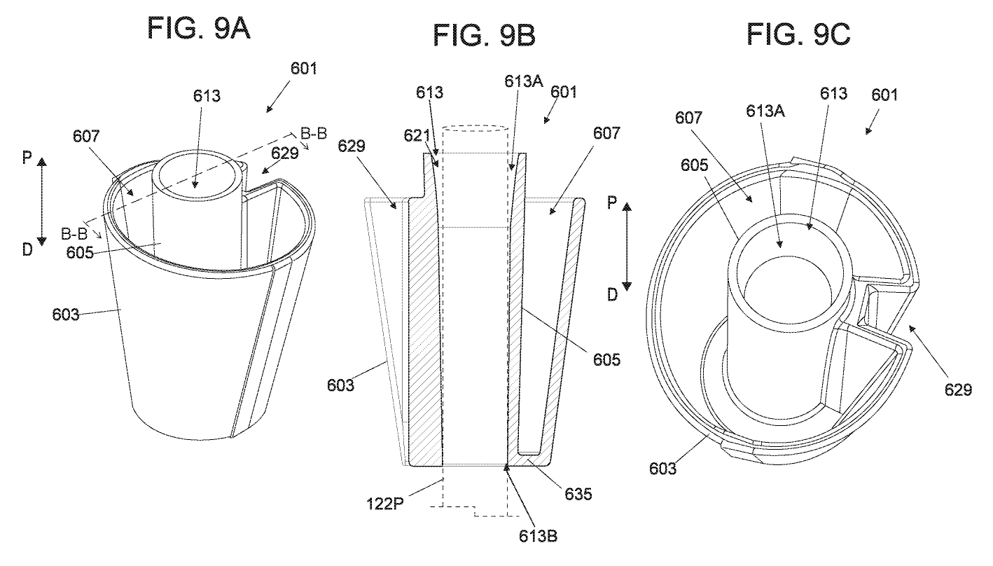

FIGS. 9A-9C illustrate an alternative hub 601. In particular, FIG. 9A illustrates a first perspective view of hub 601, FIG. 9B illustrates a cross-sectional view of hub 601, taken at the plane labeled B-B in FIG. 9A, and FIG. 9C illustrates a top perspective view of hub 601. Hub 601 may be used with handle 110 of first medical device 100. Hub 601 may have any or all of the characteristics of hub 101, hub 301, hub 401, and/or hub 501, except as described below.

In particular, hub 601 may include a first hub portion 603 and a second hub portion 605. In aspects, first hub portion 603 may be integrally formed with second hub portion 605, for example, via a distal wall 635 (FIG. 9B) extending between first hub portion 603 and second hub portion 605. A proximal-most end of second hub portion 605 may be proximal of a proximal-most end of first hub portion 603.

Hub 601 may define a fluid receptacle 607. Fluid receptacle 607 may at least partially circumferentially surround second hub portion 605. Fluid receptacle 607 may be configured to hold, or contain, fluids that leak from proximal-most portion 122P of shaft 120, working channel 158, and/or fluidics tubes 164. In these aspects, hub 601 may be configured to inhibit leaks from handle 110.

Hub 601 may include a central lumen 613 extending proximally to distally through second hub portion 605. Central lumen 613 may be configured to receive proximal-most portion 122P of shaft 120. In these aspects, second hub portion 605 may be fixed to proximal-most portion 122P of shaft 120. For ease of illustration, proximal-most portion 122P of shaft 120 is shown in broken lines only in FIG. 9B.

Central lumen 613 may include a proximal portion 613A and a distal portion 613B. Proximal portion 613A of central lumen 613 may be tapered such that a diameter of a proximal end of proximal portion 613A of central lumen 613 is greater than a diameter of a distal end of proximal portion 613A of central lumen 613. In these aspects, a gap 621 may be defined around a proximal portion of proximal-most portion 122P of shaft 120. An adhesive may be applied within gap 621 and around proximal-most portion 122P of shaft 120 such that proximal-most portion 122P of shaft 120 is fixed to hub 601. In aspects, gap 621 may be configured such that a side opening (e.g., similar to opening 127A, 127B of FIGS. 5A, 5C, 5D and 5E) is not needed. For example, adhesive may be injected into gap 621 of hub 601 as opposed to being injected through a side opening, like openings 127A, 127B of hub 101 (described above).

In aspects, a diameter of distal portion 613B of central lumen 613 may be the same or similar from a proximal end of distal portion 613B to a distal end of distal portion 613B. In other words, the walls defining distal portion 613B may be straight. In other aspects, distal portion 613B may be tapered to facilitate the insertion and/or adhesion of proximal-most portion 122P of shaft 120 to hub 601. For example, distal portion 613B of central lumen 613 may taper inward in a proximal direction (e.g., such that a diameter of the distal end of distal portion 613B of central lumen 613 is greater than a diameter of the proximal end of distal portion 613B of central lumen 613) or outward in a distal direction (e.g., such that a diameter of the distal end of distal portion 613B of central lumen 613 is smaller than a diameter of the proximal end of distal portion 613B of central lumen 613).

First hub portion 603 may further include may include a cutout 629, or a notch. Cutout 629 may have any or all of the characteristics of cutout 329 of hub 301, cutout 429 of hub 401, and/or cutout 529 of hub 501, described above. In aspects, as shown in FIG. 9B, cutout 629 may extend from a proximal-most end of first hub portion 603 to a distal-most end of first hub portion 603. In other aspects, cutout 629 may be only at a proximal portion of first hub portion 603. Cutout 629 may be configured to receive a complementary feature of handle 110, for example, to assist with the placement and/or alignment of hub 601 within handle 110.

To assemble hub 601, proximal-most portion 122P of shaft 120 may be inserted through central lumen 613 and fixed to hub 501 by injecting an adhesive into the space defined by proximal portion 613A of central lumen 613. For example, adhesive may be injected into central lumen 613 via a proximal-most opening of central lumen 613. With second hub portion 605 extending proximally relative to first hub portion 603, adhesive may more easily be injected into central lumen 613. With second hub portion 605 extending proximally relative to first hub portion 603, proximal-most portion 122P of shaft 120 may be visible from a proximal end of hub 601 (e.g., when inserted through central lumen 613). Visibility of proximal-most portion 122P of shaft 120 from the proximal end of hub 601 may allow for easier assembly. Furthermore, a length of second hub portion 605 may provide for additional surface area within central lumen 613 to which proximal-most portion 122P may be fixed (e.g., as compared to hub 701 of FIGS. 10A-10C, discussed below).

During use, fluids may leak from proximal-most portion 122P of shaft 120, working channel 158, and/or other fluidics tubes 164. The leaked fluid may be collected within fluid receptacle 607 of hub 601. Accordingly, leaks from handle 110 may be inhibited.

FIGS. 10A-10C illustrate an alternative hub 701. In particular, FIG. 10A illustrates a first perspective view of hub 701, FIG. 10B illustrates a cross-sectional view of hub 701 taken at the plane labeled B-B in FIG. 10A, and FIG. 10C illustrates a top perspective view of hub 701. Hub 701 may be used with handle 110 of first medical device 100. Hub 701 may have any or all of the characteristics of hub 101, hub 301, hub 401, hub 501, and/or hub 601, except as described below.

Hub 701 may include a first hub portion 703 and a second hub portion 705 (FIG. 10B). In aspects, first hub portion 703 may be integrally formed with second hub portion 705, for example, via a distal wall 735 (FIG. 10B) extending between first hub portion 703 and second hub portion 705. A proximal-most end of second hub portion 705 may be distal of a proximal-most end of first hub portion 703. In other words, second hub portion 705 may be only at a distal portion of hub 701.

Hub 701 may define a fluid receptacle 707. Fluid receptacle 707 may at least partially circumferentially surround second hub portion 705. With the proximal-most end of second hub portion 705 recessed as compared to the proximal-most end of first hub portion 703, fluid receptacle 707 may have an increased volume. For example, the increased volume of fluid receptacle 707 may be due to the reduction of material defining second hub portion 705. Fluid receptacle 707 may be configured to hold, or contain, fluids that leak from proximal-most portion 122P of shaft 120, working channel 158, and/or fluidics tubes 164. In these aspects, hub 701 may be configured to inhibit leaks from handle 110.

Hub 701 may include a central lumen 713 (FIG. 10B) extending proximally to distally through second hub portion 705. Central lumen 713 may be configured to receive proximal-most portion 122P of shaft 120. In these aspects, second hub portion 705 may be fixed to proximal-most portion 122P of shaft 120. For ease of illustration, proximal-most portion 122P of shaft 120 is shown in broken lines only in FIG. 10B. A proximal-most end of proximal-most portion 122P of shaft 120 may terminate or proximal of a proximal-most end of second hub portion 705. A side opening 727 may extend from an outermost surface of first hub portion 703 to an inner surface of second hub portion 705 (e.g., to an inner surface defining central lumen 713). To ensure a continuous opening from first hub portion 703 to second hub portion 705, side opening 727 may extend through a bridge portion (not shown due to the location of the cross-section defining the view of FIG. 10B), similar to bridge portion 537 of hub 501 (shown in FIG. 8C). The bridge portion may have any or all of the same characteristics of bridge portion 537 of hub 501. In these aspects, side opening 727 may be configured to permit the injection of an adhesive into central lumen 713, for example, to assist with fixing proximal-most portion 122P of shaft 120 to second hub portion 705 of hub 701 (e.g., within central lumen 713).

First hub portion 703 may include a cutout 739, or a notch, defined at or near a distal-most end of first hub portion 703. In aspects, as shown in FIG. 10C, cutout 739 may be only at or near a distal end portion of hub 701. In other aspects, cutout 739 may extend from a distal-most end of hub 701 to a proximal-most of hub 701. Cutout 739 may be configured to receive a complementary feature of handle 110, for example, to assist with the placement and/or alignment of hub 701 within handle 110.

To assemble hub 701, proximal-most portion 122P of shaft 120 may be inserted through central lumen 713 and fixed to hub 701 by injecting an adhesive into central lumen 713 via side opening 727. A proximal end of second hub portion 705 may include chambered or angled surfaces, for example, to direct the adhesive towards proximal-most portion 122P of shaft 120. The adhesive may leak, or spill, from the distal end of second hub portion 705. In aspects, spillage of adhesive from the distal end of second hub portion 705 may increase adhesion of proximal-most portion 122P of shaft 120 to second hub portion 705.

A length of second hub portion 705 may be less than an overall length of hub 701 or first hub portion 703. For example, the length of second hub portion 705 may be less than half of the overall length of hub 701 or first hub portion 703. The smaller length of second hub portion 705 as compared to hub 701 or first hub portion 703 may enable a greater volume within fluid receptacle 707. Accordingly, more fluids may be collected within fluid receptacle 707.

During use, fluids may leak from proximal-most portion 122P of shaft 120, working channel 158, and/or other fluidics tubes 164. The leaked fluid may be collected within fluid receptacle 707 of hub 701. Accordingly, leaks from handle 110 may be inhibited.

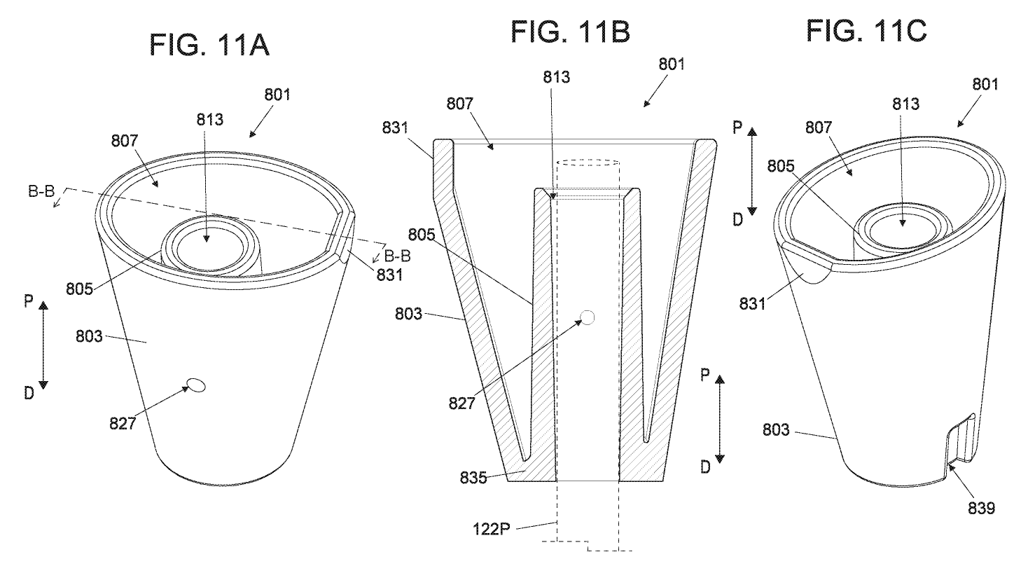

FIGS. 11A-11C illustrate an alternative hub 801. In particular, FIG. 11A illustrates a first perspective view of hub 801, FIG. 11B illustrates a cross-sectional view of hub 801 taken at the plane labeled B-B in FIG. 11A, and FIG. 11C illustrates a second perspective view of hub 801. Hub 801 may be used with handle 110 of first medical device 100. Hub 801 may have any or all of the characteristics of hub 101, hub 301, hub 401, hub 501, hub 601, and/or hub 701, except as described below.

Hub 801 may include a first hub portion 803 and a second hub portion 805. In aspects, first hub portion 803 may be integrally formed with second hub portion 805, for example, via a distal wall 835 (FIG. 10B) extending between first hub portion 803 and second hub portion 805. A proximal-most end of second hub portion 805 may be distal of a proximal-most end of first hub portion 803. As compared with hub 701 (FIGS. 10A-10C), second hub portion 805 may extend further proximally than second hub portion 705. Such a configuration may permit additional support for proximal-most portion 122P of shaft 120.

Hub 801 may define a fluid receptacle 807. Fluid receptacle 807 may at least partially circumferentially surround second hub portion 805. Fluid receptacle 807 may be configured to hold, or contain, fluids that leak from proximal-most portion 122P of shaft 120, working channel 158, and/or fluidics tubes 164. In these aspects, hub 801 may be configured to inhibit leaks from handle 110.

Hub 801 may include a central lumen 813 extending proximally to distally through second hub portion 805. Central lumen 813 may be configured to receive proximal-most portion 122P of shaft 120. In these aspects, second hub portion 805 may be fixed to proximal-most portion 122P of shaft 120. For ease of illustration, proximal-most portion 122P of shaft 120 is shown in broken lines only in FIG. 11B. A proximal-most end of proximal-most portion 122P of shaft 120 may terminate proximal of a proximal-most end of second hub portion 805. A side opening 827 may extend from an outermost surface of first hub portion 803 to an inner surface of second hub portion 805 (e.g., to an inner surface defining central lumen 813). To ensure a continuous opening from first hub portion 803 to second hub portion 805, side opening 827 may extend through a bridge portion (not shown due to the cross-section used to generate FIG. 11B), similar to bridge portion 537 of hub 501 (shown in FIG. 8C). The bridge portion may have any or all of the same characteristics of bridge portion 537 of hub 501. In these aspects, side opening 827 may be configured to permit the injection of an adhesive into central lumen 813, for example, to assist with fixing proximal-most portion 122P of shaft 120 to second hub portion 805 of hub 801 (e.g., within central lumen 813).

First hub portion 803 may include a cutout 839, or a notch, defined at or near a distal-most end of first hub portion 803. In aspects, cutout 839 may be only at or near a distal end portion of hub 801. In other aspects, cutout 839 may extend from a distal-most end of hub 801 to a proximal-most of hub 801. Cutout 839 may be configured to receive a complementary feature of handle 110, for example, to assist with the placement and/or alignment of hub 801 within handle 110. Additionally or alternatively, a proximal end of first hub portion 803 may include one or more features 831. Such features 831 may be configured to mate with complementary features of handle 110. For example, features 831 may include protrusions and/or indentations (as shown in FIGS. 11A-11C) configured to mate with complementary features of handle 110. Such features 831 may be configured to assist with the alignment and assembly of hub 101 within handle 110.

To assemble hub 801, proximal-most portion 122P of shaft 120 may be inserted through central lumen 813 and fixed to hub 801 by injecting an adhesive into central lumen 813 via side opening 827. In aspects, proximal-most portion 122P of shaft 120 may be visible from a proximal end of hub 801 when inserted through central lumen 813. Visibility of proximal-most portion 122P of shaft 120 may allow for easier assembly. Furthermore, a length of second hub portion 805 may provide for additional surface area within central lumen 813 to which proximal-most portion 122P may be fixed (e.g., as compared to hub 701 of FIGS. 10A-10C).

During use, fluids may leak from proximal-most portion 122P of shaft 120, working channel 158, and/or other fluidics tubes 164. The leaked fluid may be collected within fluid receptacle 807 of hub 801. Accordingly, leaks from handle 110 may be inhibited. With second hub portion 805 terminating proximally of a proximal-most end of first hub portion 803, fluids may more easily be captured within fluid receptacle 807.

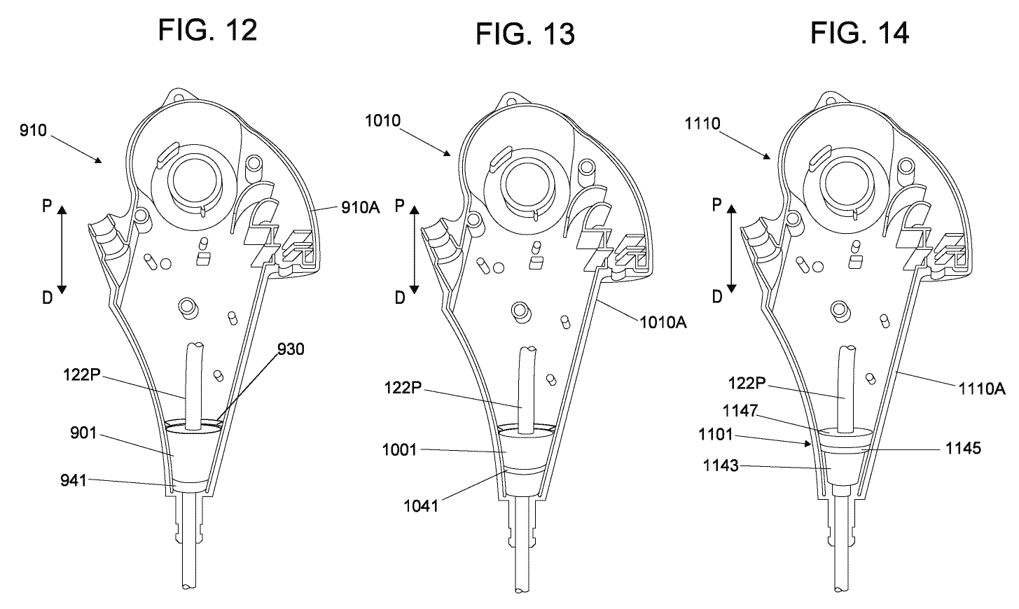

FIG. 12 illustrates an internal view of an exemplary handle 910. In particular, FIG. 12 illustrates an alternative exemplary hub 901 disposed within a first handle housing portion 910A. Hub 901 may have any or all of the characteristics of any hub 101, 301, 401, 501, 601, 701, and/or 801, previously discussed, except as described below. Similarly, handle 910, including first handle housing portion 910A, may have any or all of the characteristics of handle 110 and first handle housing portion 110A, discussed above. For example, first handle housing portion 910A may include an internal stop 930. Hub 901 may be disposed distally relative to internal stop 930. Internal stop 930 may be configured to inhibit undesired movement of hub 101 within handle 910. Internal stop 930 may have any or all of the characteristics of internal stop 130, described above with respect to FIGS. 3 and 4.

Hub 901 may be disposed within a distal portion of handle 910. Hub 901 may be overmolded onto proximal-most portion 122P of shaft 120. In other aspects, proximal-most portion 122P of shaft 120 may extend through and be fixed within a central opening of hub 901 (e.g., using an adhesive or other fixing means). Hub 901 may be a solid body, or a body without a fluid receptacle. In other aspects, hub 901 may be comprised of a flexible material, such as silicone. The flexible material may enable hub 901 to be conform against the internal walls of handle 910, thus creating a seal between hub 901 and handle 910.

A sealing member 941, such as an O-ring, a rubber-stamped gasket, or any other similar sealing device, may be disposed at a distal end of hub 901. Sealing member 941 may be disposed within a groove extending circumferentially about hub 901. The groove may assist in inhibiting movement of sealing member 941 relative to hub 901.

Sealing member 941 may be disposed between hub 901 and the internal walls of a housing of handle 910 (e.g., including first handle housing portion 110A), such that a seal is created between sealing member 941 and a housing of handle 910. In aspects, sealing member may press distally, against a distal end of handle 910 to seal the distal end. In these aspects, sealing member 941 may be configured to inhibit leaks from handle 910.

FIG. 13 illustrates an internal view of an alternative exemplary handle 1010. In particular, FIG. 13 illustrates an alternative exemplary hub 1001 disposed within a first handle housing portion 1010A. Hub 1001 may have any or all of the characteristics of any hub 101, 301, 401, 501, 601, 701, 801, and/or 901, previously discussed, except as described below. Similarly, a housing of handle 1010, including first handle housing portion 1010A, may have any or all of the characteristics of handle 110 and first handle housing portion 110A, discussed above.

Hub 1001 may be disposed within a distal portion of a housing of handle 1010. Hub 1001 may be overmolded onto proximal-most portion 122P of shaft 120. In other aspects, proximal-most portion 122P of shaft 120 may extend through and be fixed within a central opening of hub 901 (e.g., using an adhesive or other fixing means). Hub 1001 may be a solid body. A sealing member 1041, such as an O-ring or similar sealing device, may be disposed around a central portion of hub 1001. Sealing member 1041 may be disposed within a groove extending circumferentially about hub 1001. The groove may assist in inhibiting movement of sealing member 1041 relative to hub 1001. For example, movement of sealing member 1041 relative to hub 1001 may result in the seal between hub 1001 and the housing of handle 1010 to break.

Similar to sealing member 941 of FIG. 12, sealing member 1041 may be disposed between hub 1001 and the internal walls of a housing of handle 1010 (including first handle housing portion 1010A), such that a seal is created between sealing member 1041 and a housing of handle 1010. In these aspects, sealing member 1041 may be configured to inhibit leaks from handle 1010.

In some configurations, hub 1001 may have any or all of the characteristics of any hub previously discussed. For example, hub 1001 may include a fluid receptacle, similar to the fluid receptacles discussed above with respect to hubs 101, 301, 401, 501, 601, and/or 801. In addition to sealing member 1041, the fluid receptacle may assist with inhibiting leaks from handle 1010. Additional sealing members may be disposed around hub 1001. For example, similar to hub 901, hub 1001 may further include a second sealing member disposed on a distal portion of hub 1001. The additional sealing members may further assist in inhibiting leaks from handle 1010.

FIG. 14 illustrates an internal view of an alternative exemplary handle 1110. In particular, FIG. 14 illustrates an alternative exemplary hub 1101 disposed within a first handle housing portion 1110A. Hub 1101 may have any or all of the characteristics of any hub 101, 301, 401, 501, 601, 701, 801, 901, and/or 1001, previously discussed, except as described below. Similarly, handle 1110, including first handle housing portion 1110A, may have any or all of the characteristics of handle 110 and first handle housing portion 110A, discussed above.

Hub 1101 may be disposed within a distal portion of handle 1110. Hub 1101 may include a solid, distal hub portion 1143 (e.g., without a fluid receptacle). Hub 1101 may be overmolded onto or otherwise fixed to proximal-most portion 122P of shaft 120. Distal hub portion 1143 may be comprised of a polycarbonate material, or other rigid material. Hub 1101 may further include a platform 1145, or a base, fixed to or otherwise formed with distal hub portion 1143. Platform 1145 may be at a proximal portion of hub 1101. Platform 1145 may have a diameter greater than a diameter of distal hub portion 1143. Platform 1145 may be formed from a silicone or other flexible material. Accordingly, an outer diameter of platform 1145 may form a seal against the internal walls of handle 1110, for example, to assist in inhibiting leaks from handle 1110.

An absorbent disc 1147 may be disposed on top of (e.g., proximal of) platform 1145. Absorbent disc 1147 may be comprised of a porous material configured to absorb liquid. Such porous materials may include, for example, one or more of a cotton material, cellulose material, silicone material, a sponge material, a synthetic polymer, a silicone material, or any other material configured to absorb fluids. For example, as fluid leaks from proximal-most portion 122P of shaft 120, working channel 158, and/or other fluidics tubes 164, absorbent disc 1147 may absorb some or all of the fluid, thereby inhibiting leaks from handle 1110.

Distal hub portion 1143 may further include one or more sealing members (e.g., similar to sealing members 941 and/or 1041, discussed above). The sealing members may be configured to assist with inhibiting fluids from leaking from handle 1110.

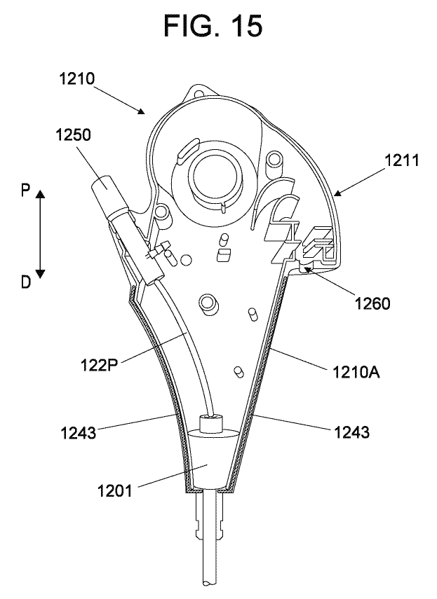

FIG. 15 illustrates an internal view of an alternative exemplary handle housing 1211 of handle 1210. In particular, FIG. 15 illustrates an alternative exemplary hub 1201 disposed within a first handle housing portion 1210A. Hub 1201 may have any or all of the characteristics of any hub 101, 301, 401, 501, 601, 701, 801, 901, 1001, and/or 1101, previously discussed, except as described below. Similarly, handle 1210, including first handle housing portion 1210A, may have any or all of the characteristics of handle 110 and first handle housing portion 110A, discussed above.

In particular, hub 1201 may be any of the hubs previously described (e.g., hub 101, 301, 401, 501, 601, 701, 801, 901,1001,1101). In these aspects, hub 1201 may be configured to inhibit leaks from handle 1210.

Additionally or alternatively to hub 1201, handle 1210 may further include a seal 1243 disposed at least partially along an edge, or a seam, for example, between each portion of handle housing 1211. For example, seal 1243 may extend distally along a portion of the seam of handle housing 1211 receiving a port 1250 to a distal end of handle housing 1211. Seal 1243 may also extend at least partially along a portion of the seal of handle housing 1211 on an opposite side of port 1250. For example, seal 1243 may extend proximally from the distal end of handle housing 1211 to an umbilicus port 1260 (e.g., a point from which an umbilicus, not shown, may extend to/from). Seal 1243 may extend along other portions of edge of handle housing 1211.

Seal 1243 may be configured to form a seal between the handle portions of handle 1210 (e.g., between first handle housing portion 1210A and a second handle portion, not shown). For example, seal 1243 may be configured to inhibit fluid from leaking from handle 1210. In aspects, seal 1243 may be an adhesive applied before, during, or after the assembly of handle 1210. In other aspects, seal 1243 may be a material fixed to, overmolded with, or otherwise integrally formed with handle housing portion 1210A (or second handle housing portion, not shown). For example, seal 1243 may be comprised of a fluid-absorbing material (e.g., a silicone, a sponge material, a synthetic polymer, or any other material configured to absorb fluids).

Unless otherwise specified, any of the hubs discussed herein (e.g., hub 101, 301, 401, 501, 601, 701 801, 901, 1001, 1101, and/or 1301) may be comprised of a polycarbonate material, or a similar rigid material. In some aspects, and of the hubs discussed herein may be molded, 3D printed, or otherwise formed. For example, any of the hubs discussed herein may be injection molded, 3D printed (including fuse deposition modeling and stereolithography), or otherwise formed. In other aspects, any of the hubs discussed herein may be comprised of a silicone or compliant material. For example, a compliant material may assist in forming additional seals between the hub and the handle by allowing for a potentially tighter fit within the handle. For example, a seal may be formed between the handle and the hub when the hub is disposed within the handle.

The devices discussed herein may include a hub disposed within a handle and configured to inhibit leaks from the handle. For example, in some aspects, the hubs discussed herein may include a fluid receptacle configured to collect fluid that leaks from one or more lumens extending through the handle. Various hub designs may be implemented, each offering different configurations of fluid receptacles and structural features. The different hub configurations discussed herein may allow for customization based on specific device requirements, potentially improving fluid management and integration with other components of the medical device. A shape of the hubs discussed herein are merely exemplary. For example, the hub may be any shape so as to fit within the handle and be retained within the handle.

A number of implementations have been described. Nevertheless, it will be understood that various modifications may be made without departing from the spirit and scope of the disclosure. Accordingly, other implementations are within the scope of the following claims.

1. A medical device handle, comprising:
a handle housing; and
a hub disposed within the handle housing, the hub comprising:
a first hub portion;
a second hub portion; and
a fluid receptacle formed between the first hub portion and the second hub portion, wherein the fluid receptacle is configured to collect fluid that leaks from one or more fluid lumens extending at least partially through the handle housing.

2. The medical device handle of claim 1, wherein the hub further comprises a lumen through the second hub portion.

3. The medical device handle of claim 2, wherein the lumen is configured to receive the one or more fluid lumens.

4. The medical device handle of claim 2, wherein a proximal portion of the lumen tapers from a proximal end to a distal end of the proximal portion.

5. The medical device handle of claim 2, further comprising an opening extending from an outer surface of the first hub portion to an inner surface of the lumen.

6. The medical device handle of claim 5, wherein the opening extends through a bridge disposed between the first hub portion and the second hub portion.

7. The medical device handle of claim 1, wherein the first hub portion includes at least one cutout, wherein the at least one cutout is configured to receive a complementary feature of the handle housing.

8. The medical device handle of claim 1, wherein the second hub portion further comprises a cylindrical portion, and wherein a first wing and a second wing extend proximally from the cylindrical portion.

9. The medical device handle of claim 8, wherein the first hub portion includes a first groove and a second groove each extending along an inner surface of the first hub portion, wherein the first groove is configured to receive the first wing, and wherein the second groove is configured to receive the second wing.

10. The medical device handle of claim 8, wherein a width of the first wing is smaller than a width of the second wing.

11. The medical device handle of claim 8, wherein a rib is disposed on a proximal-most end of each of the first wing and the second wing.

12. The medical device handle of claim 1, wherein the second hub portion includes a proximal cylindrical portion and a distal cylindrical portion, wherein an inner diameter of the proximal cylindrical portion is greater than an inner diameter of the distal cylindrical portion.

13. The medical device handle of claim 1, wherein a proximal-most end of the first hub portion is distal of or adjacent to a proximal-most end of the second hub portion.

14. The medical device handle of claim 1, wherein an internal surface of the handle housing includes an internal stop, wherein a distal end of the internal stop is configured to abut a proximal-most end of the first hub portion.

15. The medical device handle of claim 1, wherein the first hub portion comprises a frustoconically-shaped outer surface.

16. A hub disposed within a handle of a medical device, the hub comprising:
a first hub portion;
a second hub portion disposed within the first hub portion, the second hub portion comprising a lumen extending through the second hub portion, wherein the lumen is configured to receive a tubular member comprising at least one lumen; and
a fluid receptacle formed between the first hub portion and the second hub portion, wherein the fluid receptacle is configured to collect fluid that leaks from the tubular member.

17. The hub of claim 16, further comprising at least one opening extending from an outer surface of the first hub portion to an inner surface of the lumen of the second hub portion.

18. The hub of claim 16, wherein the second hub portion further comprises a cylindrical portion, wherein a first wing and a second wing extend proximally from the cylindrical portion.

19. The hub of claim 18, wherein the first hub portion includes a first groove and a second groove each extending along an inner surface of the first hub portion, wherein the first groove is configured to receive the first wing, and wherein the second groove is configured to receive the second wing.

20. A medical device handle, comprising:
a handle housing; and
a hub disposed within the handle housing, and wherein the hub comprises:
a first hub portion having a first wall;
a second hub portion disposed within the first hub portion, the second hub portion having a second wall, wherein the second wall defines a lumen extending through the second hub portion, and wherein the lumen is configured to receive a tubular member comprising at least one lumen, and wherein a gap is defined between the first wall and the second wall; and
an adhesive disposed between the tubular member and the second wall.
